(19) United States
(12) Patent Application Publication
(10) Pub. No.: US 2013/0344001 A1
(43) Pub. Date: Dec. 26, 2013

(54) INDOCYANINE GREEN-CONTAINING PARTICLES, PHOTOACOUSTIC-IMAGING CONTRAST AGENT INCLUDING THE SAME, AND METHOD FOR PRODUCING THE INDOCYANINE GREEN-CONTAINING PARTICLES

(71) Applicant: CANON KABUSHIKI KAISHA, Tokyo (JP)

(72) Inventors: Sachiko Yamauchi, Yokohama-shi (JP); Fumio Yamauchi, Kyoto-shi (JP); Mayuko Kishi, Machida-shi (JP); Yoshinori Tomida, Atsugi-shi (JP)

(21) Appl. No.: 13/922,152

(22) Filed: Jun. 19, 2013

(30) Foreign Application Priority Data
Jun. 20, 2012 (JP) 2012-138393

(51) Int. Cl. A61K 49/22

(52) U.S. Cl. CPC A61K 49/222
USPC 424/9.5

(57) ABSTRACT

Indocyanine green-containing particles each include a metal oxide particle or a metal particle and an aggregate of indocyanine green. The aggregate of indocyanine green has a relative maximum absorbance at 880 nm or more and 910 nm or less.

### BACKGROUND OF THE INVENTION

1. Field of the Invention

The present invention relates to indocyanine green-containing particles, a photoacoustic-imaging contrast agent including the indocyanine green-containing particles, and a method for producing the indocyanine green-containing particles.

2. Description of the Related Art

Photoacoustic imaging method is a known technique for visualizing the inside of a living body. The photoacoustic imaging method is used to obtain the distribution of materials inside an analyte irradiated with light as an image by determining the intensity of an acoustic wave (photoacoustic signal) generated from the analyte and the position at which the acoustic wave is generated.

It is known that particles prepared by coating iron oxide particles with dextran (Resovist, registered trademark) absorb light and generate an acoustic wave (Analytical Chemistry 2005; 77: pp. 2381-2385, hereinafter referred to as “Non-Patent Document 1”). Thus, Resovist can be used as a photoacoustic-imaging contrast agent. It is also known that indocyanine green (hereinafter abbreviated as “ICG”) absorbs light and generates an acoustic wave. When an aggregate of ICG is not formed, ICG has a relative maximum absorbance in a wavelength band around 780 nm. Note that the term “ICG” herein refers to a compound having the structure illustrated below:

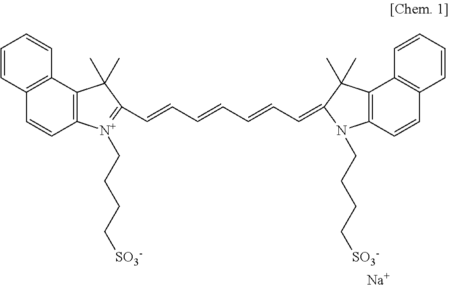

where the counter ion is not limited to Na⁺ and may be any counter ion such as H⁺ or K⁺.

Particles prepared by combining iron oxide particles and ICG, which both absorb light and generate an acoustic wave, may generate a strong acoustic wave. Non-Patent Document 1 discloses a particle that is a gold particle having ICG adsorbed thereon (hereinafter referred to as “ICG-gold probe”). In Non-Patent Document 1, the ICG-gold probe is used for surface-enhanced Raman scattering. However, gold particles and ICG, which both absorb light and generate an acoustic wave, may generate a strong acoustic wave.

The inventors have conducted extensive studies and, as a result, found that the ICG-gold probe disclosed in Non-Patent Document 1 has a problem. Specifically, when the ICG-gold probe is placed in water, highly hydrophilic sulfonate groups of ICG interact with water molecules, and thereby ICG may be desorbed from the ICG-gold probe because ICG is considered to be adsorbed on gold particles with a weak interaction. In addition, when the ICG-gold probe is placed in blood serum, ICG interacts with proteins present in blood serum, such as albumen, and thereby ICG may be desorbed from the ICG-gold probe.

Accordingly, the present invention provides particles in which ICG is less likely to be desorbed from metal oxide particles or metal particles.

### SUMMARY OF THE INVENTION

Indocyanine green-containing particles according to an embodiment of the invention each include a metal oxide particle or a metal particle; and an aggregate of indocyanine green. The aggregate of indocyanine green has a relative maximum absorbance at 880 nm or more and 910 nm or less.

A method for producing the indocyanine green-containing particles according to an embodiment of the invention includes the steps of:

heating an aqueous solution of indocyanine green;
mixing the heated aqueous solution of indocyanine green with metal oxide particles or metal particles to prepare a liquid mixture; and
heating the liquid mixture.

Further features of the present invention will become apparent from the following description of exemplary embodiments with reference to the attached drawings.

### BRIEF DESCRIPTION OF THE DRAWINGS

FIG. 1 is a schematic diagram illustrating the cross section of an indocyanine green-containing particle according to an embodiment of the invention.

FIGS. 2A and 2B are diagrams illustrating the structure of ICG.

FIGS. 3A and 3B are diagrams for explaining the reason why ICG is less likely to be desorbed from indocyanine green-containing particles according to an embodiment of the invention.

FIG. 4 is a diagram showing the results of the measurement of the absorbances of indocyanine green-containing particles prepared in Examples of the invention.

FIG. 5 is a diagram showing the results of the measurement of the absorbances of other indocyanine green-containing particles prepared in Examples of the invention.

FIGS. 6A and 6B are diagrams showing the results of the evaluation of indocyanine green-containing particles prepared in Examples and Comparative examples of the invention in terms of stability in water.

FIG. 7 is a diagram showing the results of the evaluation of indocyanine green-containing particles prepared in Examples of the invention in terms of storage stability in blood serum.

FIG. 8 is a diagram showing the results of the measurement of the absorbances of indocyanine green-containing particles prepared in Comparative examples of the invention.

### DESCRIPTION OF THE EMBODIMENTS

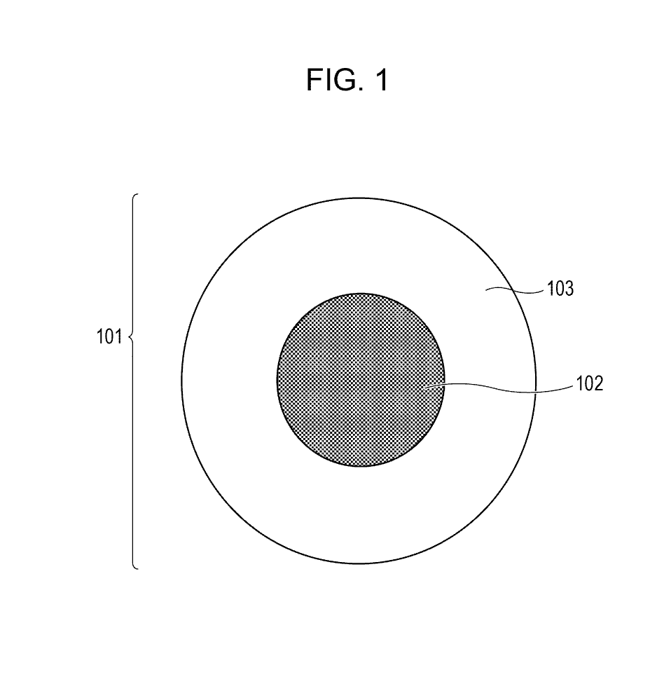

An embodiment of the invention will now be described with reference to FIG. 1, which illustrates the cross section of indocyanine green-containing particles according to the embodiment of the invention. Hereinafter, the term “indocyanine green-containing particles” may be referred to as simply “particles”.

### Particles

Particles 101 according to the embodiment each include a metal oxide particle or a metal particle 102 and an aggregate of ICG 103. The aggregate of ICG 103 has a relative maximum absorbance at 880 nm or more and 910 nm or less.

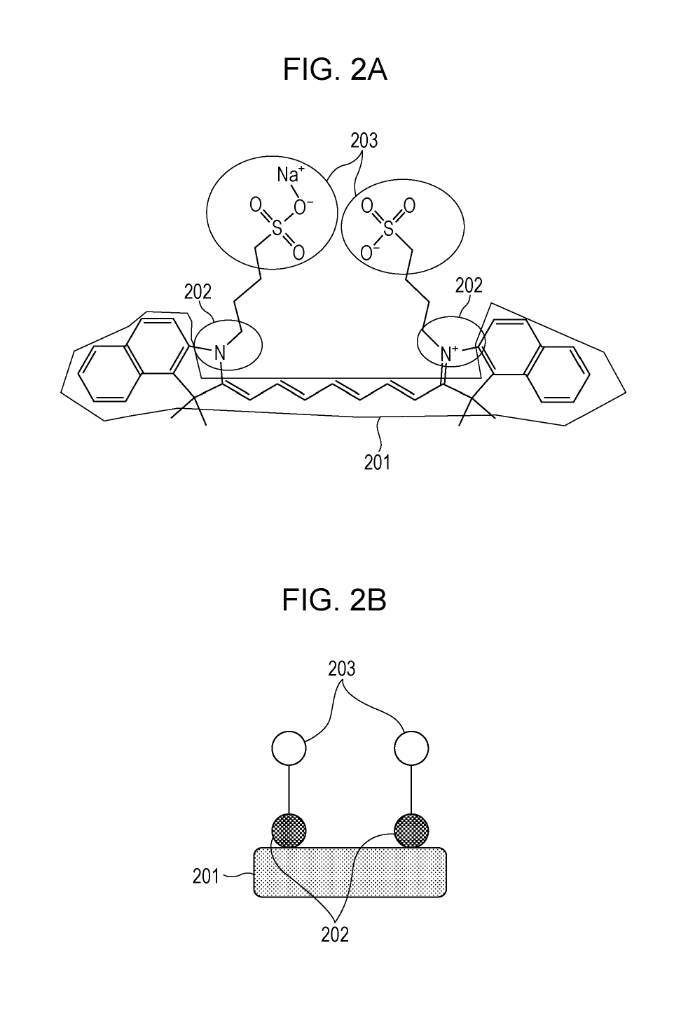

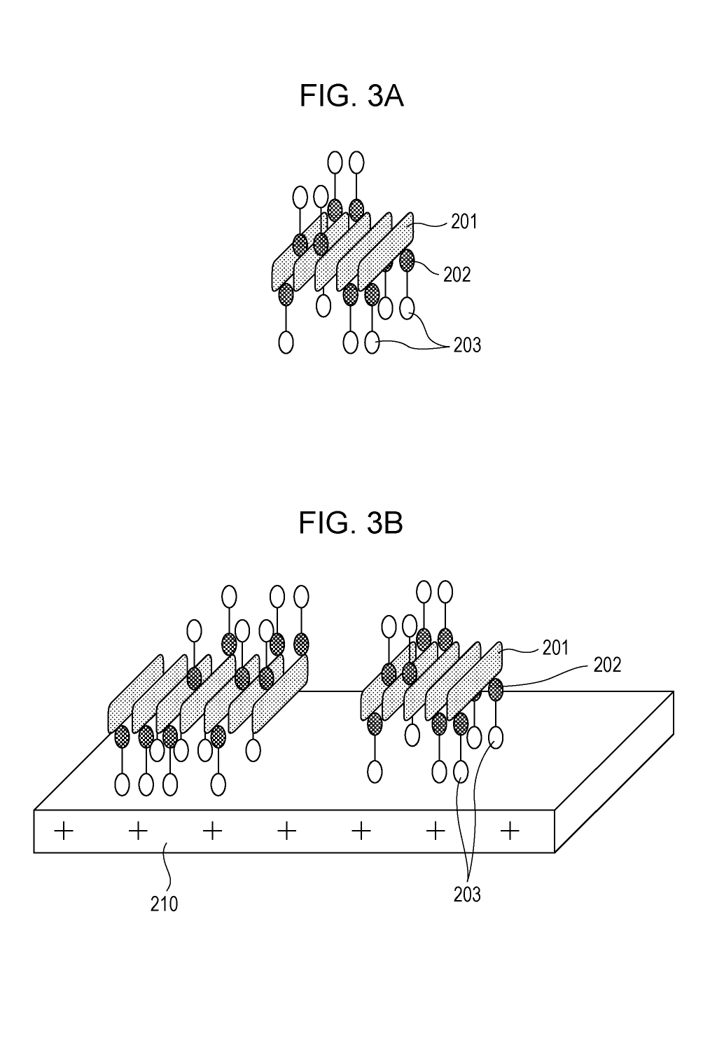

In the particles 101 according to the embodiment, ICG is less likely to be desorbed from metal oxide particles or metal particles even when the particles 101 are placed in water or blood serum. The reason for this will now be described with reference to FIGS. 2A to 3B.

FIG. 2A illustrates the structural formula of ICG, which may be divided into three portions: a hydrophobic portion 201 including aromatic rings and a methine chain, a hydrophilic portion 203 including sulfonate groups, and an intermediate portion 202 between the hydrophobic portion 201 and the hydrophilic portion 203. FIG. 2B schematically illustrates the structure of ICG shown in FIG. 2A.

Negatively charged sulfonate groups of the hydrophilic portion 203 of ICG and a positively charged metal oxide particle or metal particle interact with each other, and thereby ICG is adsorbed onto the metal oxide particle or the metal particle. However, ICG may be desorbed from the metal oxide particle or the metal particle when the particles 101 are placed in water or blood serum because ICG is adsorbed on the metal oxide particle or the metal particle with a weak interaction.

This is presumably because water molecules, which have a high affinity for the sulfonate groups of the hydrophilic portion 203, interact with the sulfonate groups adsorbed on the metal oxide particle or the metal particle, and thereby ICG is desorbed from the metal oxide particle or the metal particle. In addition, proteins in blood serum, such as albumen, may interact with ICG, and thereby ICG is desorbed from the metal oxide particle or the metal particle.

The hydrophobic portion 201 of ICG may be stacked on top of one another to form an aggregate composed of a plurality of ICG molecules (FIG. 3A). FIG. 3A schematically illustrates the structure of ICG shown in FIG. 2B forming an aggregate. The relative maximum absorbance of ICG forming an aggregate differs from the relative maximum absorbance of ICG not forming an aggregate. ICG according to the embodiment has a relative maximum absorbance at 880 nm or more and 910 nm or less and may be referred to as “J-aggregate of ICG”. In FIG. 3B, the surface of the metal oxide particle or the metal particle is schematically illustrated as a portion 210, on which an aggregate of ICG is adsorbed. The aggregate of ICG, which is composed of a plurality of ICG molecules as shown in FIG. 3A, has more sulfonate groups than a single ICG molecule. Thus, the aggregate of ICG may interact with the metal oxide particle or the metal particle (portion 210) at a plurality of points and thereby be adsorbed thereon as shown in FIG. 3B. In other words, the aggregate of ICG may be adsorbed on the metal oxide particle or the metal particle at a plurality of points while maintaining the connections among the ICG molecules, whereas the single ICG molecule is adsorbed on the metal oxide particle or the metal particle at two points at maximum, which is the number of the sulfonate groups per ICG molecule. Therefore, the aggregate of ICG is less likely to be desorbed from the metal oxide particle or the metal particle (FIG. 3B).

In the aggregate of ICG, the ICG molecules may be stacked on top of one another with the hydrophilic sulfonate groups of the hydrophilic portion 203 being oriented in opposite directions as shown in FIG. 3A; some sulfonate groups of the hydrophilic portion 203 oriented in a certain direction are adsorbed on a metal oxide particle or a metal particle and the other sulfonate groups oriented in the opposite direction face toward the outside of the particle as shown in FIG. 3B. When the hydrophilic sulfonate groups face toward the outside of the particle, water molecules may interact with the sulfonate groups facing toward the outside of the particle, and thereby ICG may easily be removed from an aggregate of ICG adsorbed on the metal oxide particle or the metal particle. However, the ICG molecules are less likely to come in contact with water molecules than the single ICG molecule because the ICG molecules are stacked on top of one another, that is, the ICG molecules are in contact with one another. Water molecules are less likely to approach the ICG molecules and therefore less likely to remove the ICG molecules. As a result, the ICG molecules are less likely to be desorbed from the metal oxide particle or the metal particle. As is described above, when an aggregate of ICG is adsorbed on a metal oxide particle or a metal particle, ICG molecules are adsorbed on the metal oxide particle or the metal particle at a plurality of points and stacked on top of one another, thereby being less likely to be approached by water molecules. Therefore, the ICG molecules are less likely to be desorbed from the metal oxide particle or the metal particle even in water. When the particles according to the embodiment are placed in blood serum, similarly, proteins in blood serum having a high affinity for water, such as albumen, are less likely to approach ICG molecules because the ICG molecules are stacked on top of one another. Even if such proteins in blood serum could approach the ICG molecules, proteins such as albumen are less likely to remove the ICG molecules, which are adsorbed on the metal oxide particle or the metal particle at a plurality of points. Thus, in the particles according to the embodiment, ICG is less likely to be desorbed from a metal oxide particle or a metal particle even when the particles are placed in water or blood serum. Adsorption herein refers to bonding between an aggregate of ICG and a metal oxide particle or a metal particle due to Coulomb interaction, which is, specifically, caused by negatively charged sulfonate groups of an aggregate of ICG and positively charged metal atoms of a metal oxide particle or a metal particle.

The absorbance of the particles according to the embodiment at 895 nm is preferably 2.0 times or more and more preferably 2.5 times or more the absorbance at 780 nm. This is because, when the absorbance at 895 nm is more than the absorbance at 780 nm, the proportion of the aggregates of ICG in the particles is considered to be large.

The particles according to the embodiment may each include the aggregate of ICG inside the particle.

The particles according to the embodiment may each include a dispersant on the surface of the particle.

### Metal Oxide Particles or Metal Particles

In this embodiment, the metal oxide particles or the metal particles are not particularly limited as long as an aggregate of ICG can be adsorbed thereon. Examples of the metal oxide particles include iron oxide particles, aluminum oxide particles, magnesium oxide particles, titanium oxide particles, copper oxide particles, zinc oxide particles, manganese oxide particles, cobalt oxide particles, nickel oxide particles, tin oxide particles, cerium oxide particles, and calcium oxide particles. Examples of the metal particles include gold particles, silver particles, copper particles, and platinum particles.

### Iron Oxide Particles

In this embodiment, the metal oxide particles are preferably iron oxide particles, which absorb near-infrared light and are confirmed to be safe for living bodies.

In this embodiment, examples of the iron oxide particles include Fe₃O₄ (magnetite) particles, γ-Fe₂O₃ (maghemite) particles, and mixtures thereof. Magnetite is preferably used because it is known to have a higher molar absorptivity than maghemite in the near-infrared region, which may accordingly increase the intensity of the acoustic wave generated upon irradiation with light. In this embodiment, the iron oxide particles may be in a single-crystal, polycrystalline, or amorphous state. When the particles according to the embodiment are used to prepare an MRI imaging contrast agent, the iron oxide particles are preferably in a single-crystal state.

In this embodiment, the iron oxide particles may optionally be magnetic. When the iron oxide particles are magnetic, the particles can be purified using a magnet.

### Aggregate of ICG

The aggregate of ICG according to the embodiment has a relative maximum absorbance at 880 nm or more and 910 nm or less, preferably at 890 nm or more and 900 nm or less, and more preferably at 895 nm. The aggregate of ICG may be formed by heating an aqueous solution of ICG.

### Dispersant

In this embodiment, a dispersant is used to improve the dispersibility of the particles according to the embodiment in an aqueous solution such as water or blood serum. The dispersant is preferably used particularly when iron oxide particles, which easily aggregate with one another, are used.

Examples of the dispersant according to the embodiment include polysaccharides, tetrasaccharides, trisaccharides, disaccharides, monosaccharides, sugar alcohols, and polyethylene glycol (PEG).

Examples of the polysaccharides include dextran, carboxydextran, aminodextran, dextrin, sodium hyaluronate, pullulan, alginic acid, pectin, amylopectin, glycogen, cellulose, agarose, carrageenan, heparin sodium, xyloglucan, and xanthan gum.

Examples of the tetrasaccharides include acarbose and stachyose.

Examples of the trisaccharides include raffinose, melezitose, and maltotriose.

Examples of the disaccharides include trehalose, sucrose, lactose, maltose, turanose, and cellobiose.

Examples of the monosaccharides include dihydroxyacetone, glyceraldehyde, erythrose, erythrulose, threose, ribulose, xylulose, xylose, lyxose, deoxyribose, psicose, fructose, sorbose, tagatose, amylose, glucose, mannose, gulose, galactose, talose, fucose, rhamnose, and sedoheptulose.

Examples of the sugar alcohols include xylitol, inositol, calcium gluconate, sodium gluconate, magnesium gluconate, sorbitol, calcium saccharate, hydroxypropyl cellulose, mannitol, and meglumine.

PEG may optionally chemically bond to a saccharide listed above.

PEG may be linear or branched.

### Particle Size

Particle size herein refers to hydrodynamic diameter measured by dynamic light scattering using a dynamic light scattering analyzer. The size of the particles according to the embodiment is preferably 1 nm or more and 5,000 nm or less. The size of the particles according to the embodiment is more preferably 8 nm or more, at which the particles are less likely to be excreted from kidneys, and 1,000 nm or less, at which an enhanced permeability and retention (EPR) effect is expected.

### Applications

The particles according to the embodiment may suitably be used as a photoacoustic-imaging contrast agent because ICG is less likely to be desorbed from the particles and thereby a strong acoustic wave can be generated.

The particles according to the embodiment may also be used as an MRI contrast agent.

### Photoacoustic-Imaging Contrast Agent

The photoacoustic-imaging contrast agent according to the embodiment includes the particles according to the embodiment and a dispersion medium. The concept of “photoacoustic imaging” herein includes photoacoustic tomography. Examples of the dispersion medium include physiological saline, distilled water for injections, phosphate buffered saline, and an aqueous solution of glucose. The photoacoustic-imaging contrast agent according to the embodiment may optionally include pharmacologically allowable additives such as vasodilators.

The photoacoustic-imaging contrast agent according to the embodiment may be dispersed in the dispersion medium in advance, or may be prepared in the form of a kit and dispersed in the dispersion medium before administration into a living body.

The photoacoustic-imaging contrast agent according to the embodiment, due to the EPR effect, can accumulate at tumor sites more than at normal sites in a living body when administered into the living body. As a result, when the particles are administered into a living body and subsequently the living body is irradiated with light and generates an acoustic wave, the intensity of the acoustic wave generated from the tumor sites may become greater than that generated from the normal sites. Thus, the particles according to the embodiment may be used as a photoacoustic-imaging contrast agent used for detecting specifically a tumor site.

### Photoacoustic-Imaging Method

The photoacoustic-imaging method according to the embodiment includes the steps of irradiating an analyte in which the indocyanine green-containing particles or photoacoustic-imaging contrast agent is administered with a light having a wavelength of 600 nm to 1,300 nm and detecting an acoustic wave generated from the contrast agent present inside the analyte.

An example of the photoacoustic-imaging method according to the embodiment is described below. The indocyanine green-containing particles or contrast agent according to the embodiment is administered by an analyte or added to a sample, such as an organ, taken from the analyte. The analyte is not particularly limited and examples thereof include, in addition to a human, mammals such as laboratory animals and pets. Examples of the sample taken from the analyte include organs, tissues, tissue sections, cells, and cell lysates. After the administration or addition of the contrast agent according to the embodiment, the analyte or the like is irradiated with a near-infrared laser pulse.

The photoacoustic signal (acoustic wave) generated from the indocyanine green-containing particles or contrast agent according to the embodiment is then detected with an acoustic wave detector, such as a piezoelectric transducer, and converted into an electric signal. The position or size of an absorbent in the analyte and the distribution of optical property, such as absorption coefficient, may be estimated on the basis of the electric signal detected with the acoustic wave detector. For example, when a photoacoustic signal having an intensity exceeding a reference threshold is detected, the following reasons are considered: the analyte contains target molecules or a site that produces the target molecules; the sample contains the target molecules; or the analyte from which the sample is taken contains a site that produces the target molecules.

### Method for Producing Particles

The method for producing particles according to the embodiment is not particularly limited and examples of the method include a method in which an aggregate of ICG is prepared and the aggregate of ICG is then adsorbed onto a metal oxide particle or a metal particle and a method in which ICG adsorbed on a metal oxide particle or a metal particle is formed into an aggregate.

An example of the method for producing particles according to the embodiment includes the following Steps 1 to 3, which are performed in this order.

Step 1: Heating an aqueous solution of ICG
Step 2: Mixing the heated aqueous solution of ICG with metal oxide particles or metal particles
Step 3: Heating a liquid mixture prepared in Step 2
According to the study conducted by the inventors, a change in relative maximum absorbance, which indicates the formation of an aggregate, was not observed when the particles were prepared through Steps 1 to 3 with ICG concentration of 3.0×10⁻⁷ mol/ml. According to Chemical Physics 1997; 220: pp. 385-392, when an aqueous solution of ICG with a concentration of 10⁻⁶ mol/ml or more is heated, the wavelength at which the relative maximum absorbance is observed, which is in the range of 700 to 800 nm, shifts to 890 nm, and this was demonstrated at a concentration of 1.5×10⁻⁶ mol/ml. Thus, the ICG concentration in Step 1 is preferably 10⁻⁶ mol/ml or more and less than 3.0×10⁻⁷ mol/ml and more preferably 1.5×10⁻⁶ mol/ml or more. For example, when the heated aqueous solution of ICG is mixed with an aqueous dispersion of iron oxide particles in a volumetric ratio of 9:1 in Step 2, the concentration of the aqueous solution of ICG in Step 1 is preferably adjusted to be about 1.7×10⁻⁶ mol/ml.

The heating temperature in Steps 1 and 3 is preferably 40° C. or more because a temperature of 40° C. or more shortens the time required to form an aggregate of ICG. The time of heating the aqueous solution of ICG in Step 1 is preferably 1 hour or more and 24 hours or less.

According to the study conducted by the inventors, even when an aqueous solution of ICG was mixed with the aqueous dispersion of iron oxide particles and the resulting liquid mixture was heated, ICG was desorbed from the iron oxide particles when particles designed for administration into living bodies were mixed with blood serum. Therefore, heating the aqueous solution of ICG, Step 1, needs to be performed before the addition of the aqueous dispersion of iron oxide particles to the aqueous solution of ICG.

### EXAMPLES

The invention will be described in detail with reference to Examples, which do not limit the scope of the invention. Materials, compositions, reaction conditions, and the like may be modified in various ways as long as particles having similar functions and effects can be produced.

### Example 1

### Preparation of Particles Each Including Iron Oxide Particle and Aggregate of ICG (Particles A-1 to A-3)

Particles each including an iron oxide particle and an aggregate of ICG (particles A-1 to A-3) were prepared as follows.

Water was added to ICG (indocyanine green reference standard, produced by Pharmaceutical and Medical Device Regulatory Science Society of Japan) to prepare a liquid mixture having an ICG concentration of 1.29 mg/ml. The liquid mixture was then irradiated with an ultrasonic wave for 10 minutes to dissolve ICG in water. The resulting aqueous solution of ICG was divided into 3 portions, which were then heated at 65° C. for 1, 3, and 6 hours. The heated aqueous solution of ICG was mixed with an aqueous solution in which iron oxide particles (Nanomag 45-00-202, γ-Fe₂O₃, produced by Corefront Corporation) were dispersed in a volumetric ratio of 9:1. The size of iron oxide particles used was 1,338 nm.

The liquid mixture of the aqueous solution of ICG and the aqueous solution in which the iron oxide particles were dispersed was then heated at 65° C. for 24 hours. The iron oxide particles were collected using a magnet to remove ICG that was not adsorbed on the iron oxide particles. Particles prepared by heating the aqueous solution of ICG for 1 hour, 3 hours, and 6 hours were denoted as A-1, A-2, and A-3, respectively.

The sizes of particles A-1 to A-3 were measured using a dynamic light scattering analyzer (ELS-Z, produced by Otsuka Electronics Co., Ltd.). For each of particles A-1 to A-3, the measurement was repeated 100 times, this was repeated for 5 times to obtain 5 particles sizes, and these particle sizes were averaged to obtain the average size of the particles. The average sizes of particles A-1, A-2, and A-3 were 482 nm, 432 nm, and 419 nm, respectively.

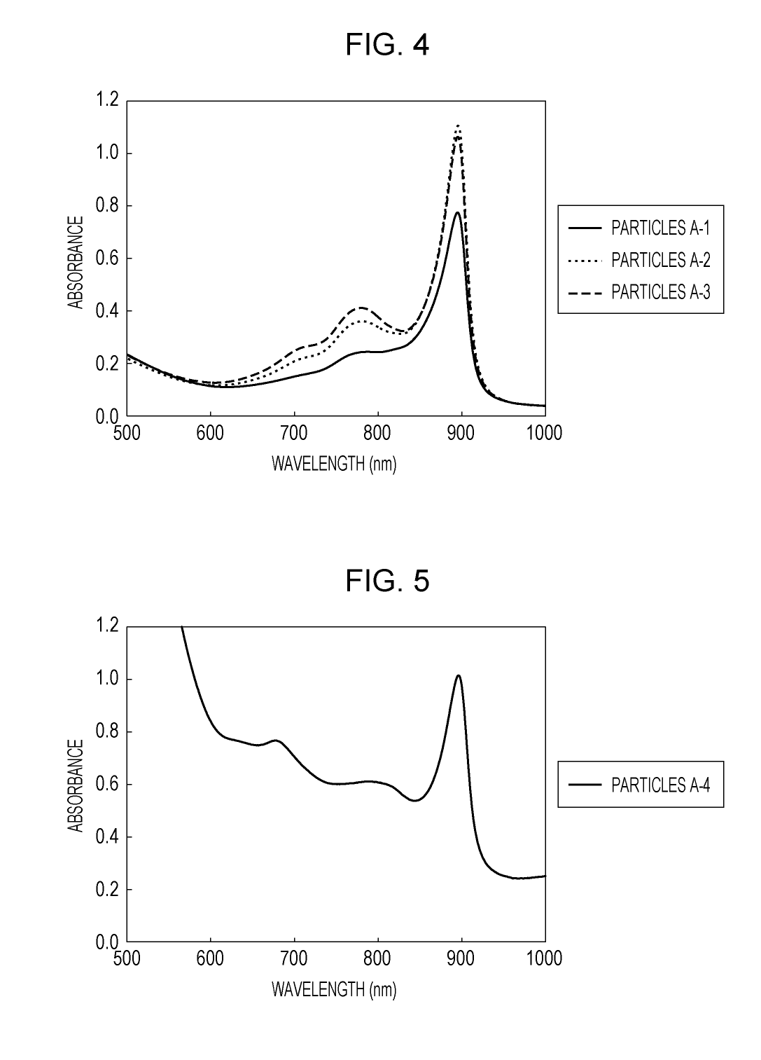

For each of particles A-1, A-2, and A-3, the absorbance was determined using a spectrophotometer (Perkin Elmer Lambda Bio40). FIG. 4 shows the measurement results. All of particles A-1, A-2, and A-3 had a relative maximum absorbance at 895 nm. The determined absorbance was presumably due to ICG because it is known that iron oxide particles hardly absorb a light having a wavelength of 895 nm. Thus, it was found that a J-aggregate of ICG was formed in Example 1.

In particles A-1, A-2, and A-3, the ratios of the absorbance at 895 nm to the absorbance at 780 nm were 3.2, 3.1, and 2.6, respectively.

### Example 2

Preparation of Particles Each Including Iron Oxide Particle and Aggregate of ICG with Dispersant on Surface of Particle (Particles A-4)

Particles each including an iron oxide particle and an aggregate of ICG with dextran serving as a dispersant on the surface of the particle (particles A-4) were prepared as follows.

Particles A-4 were prepared as in Example 1, except that iron oxide particles (Nanomag 79-00-501, particle size: 50 nm, γ-Fe₂O₃, produced by Corefront Corporation) with dextran on the surfaces of the particle were used instead of the iron oxide particles used in Example 1 (Nanomag 45-00-202, γ-Fe₂O₃, produced by Corefront Corporation). In addition, the time for heating the aqueous solution of ICG in Step 1 was 6 hours. The iron oxide particles were collected using a magnetic column (MACS, produced by Miltenyi Biotec K.K.) to remove ICG that was not adsorbed on the iron oxide particles. The resulting particles are denoted as A-4.

The size and absorbance of particles A-4 were determined using a dynamic light scattering analyzer as in the measurements of particles A-1, A-2, and A-3. The average particle size was 96 nm. FIG. 5 shows the results of the measurement of the absorbance of particles A-4. The relative maximum absorbance was observed at 895 nm. The determined absorbance was presumably due to ICG because it is known that iron oxide particles and dextran hardly absorb a light having a wavelength of 895 nm. Thus, it was found that a J-aggregate of ICG was formed in Example 2. The ratio of the absorbance at 895 nm to the absorbance at 780 nm was 2.7.

### Evaluation of Particles A-3 in Terms of Stability in Water

The particles A-3 was evaluated in terms of stability in water as follows.

An aqueous dispersion of particles A-3 was condensed using a magnet so as to have an absorbance of about 200. A portion of the condensed aqueous dispersion was taken and diluted 200-fold, and the absorbance thereof was determined. The remaining portion of the condensed aqueous dispersion was left at a room temperature for 1 day and subsequently diluted 200-fold, and the absorbance thereof was determined.

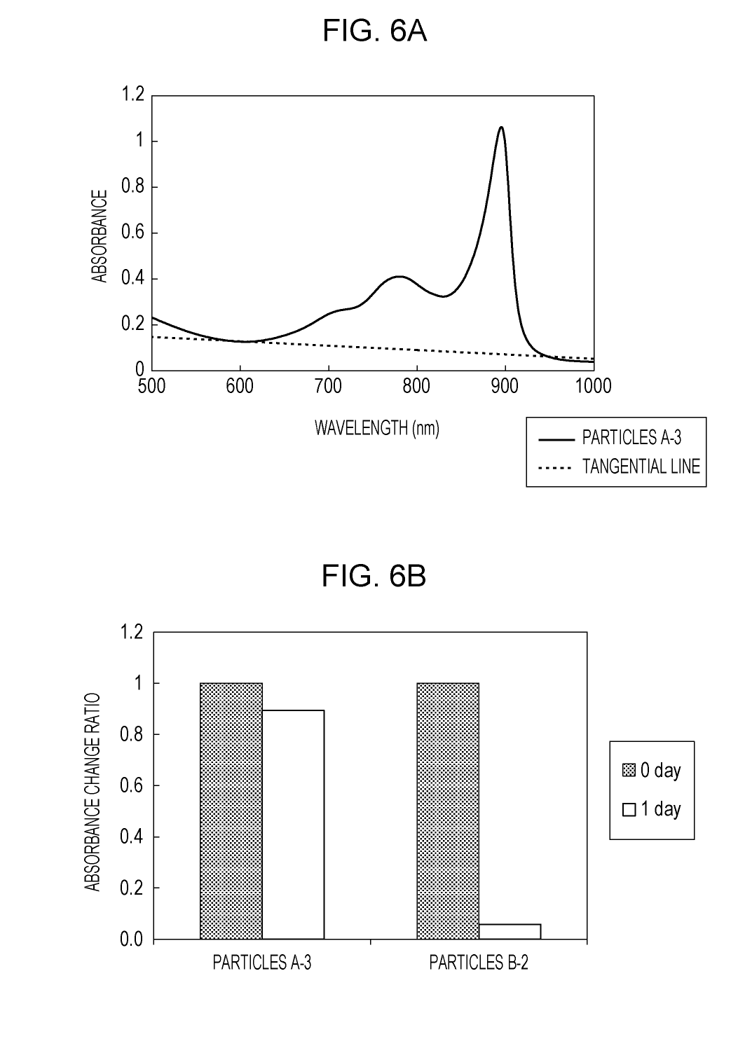

FIG. 6A shows the results of the measurement of the absorbance of particles A-3. Since no peak absorbance of ICG was observed at a wavelength of about 600 nm and at a wavelength of about 950 nm, a line tangential to the absorbance curve at 600 nm and at 950 nm was drawn as shown in FIG. 6A, and the difference between the tangential line and the absorption of particles A-3 was determined to be the absorption due to the aggregate of ICG. In the case where the absorbance (absorbance change ratio) due to an aggregate of ICG contained in particles before being left for 1 day is considered to be 1, the absorbance due to an aggregate of ICG contained in particles after being left for 1 day is determined. The absorbance due to an aggregate of ICG contained in particles A-3 was determined at a wavelength of 895 nm.

FIG. 6B shows the absorbance change ratio of particles A-3. It was found that, in particles A-3, which had a small absorbance change ratio, ICG was less likely to be desorbed from the iron oxide particles in water.

### Evaluation of Particles A-1 to A-4 in Terms of Stability in Blood Serum

As a model experiment for demonstrating the administration of particles A-1 to A-4 into a living body, particles A-1 to A-4 were each mixed with blood serum and the stability of the particles was determined as follows.

Aqueous dispersions of particles A-1 to A-4 were each mixed with blood serum in a volumetric ratio of 1:9, and immediately the absorbance of the mixture was determined.

After the absorbance was determined, the mixture was incubated at 37° C. for 24 hours, and the absorbance was determined again.

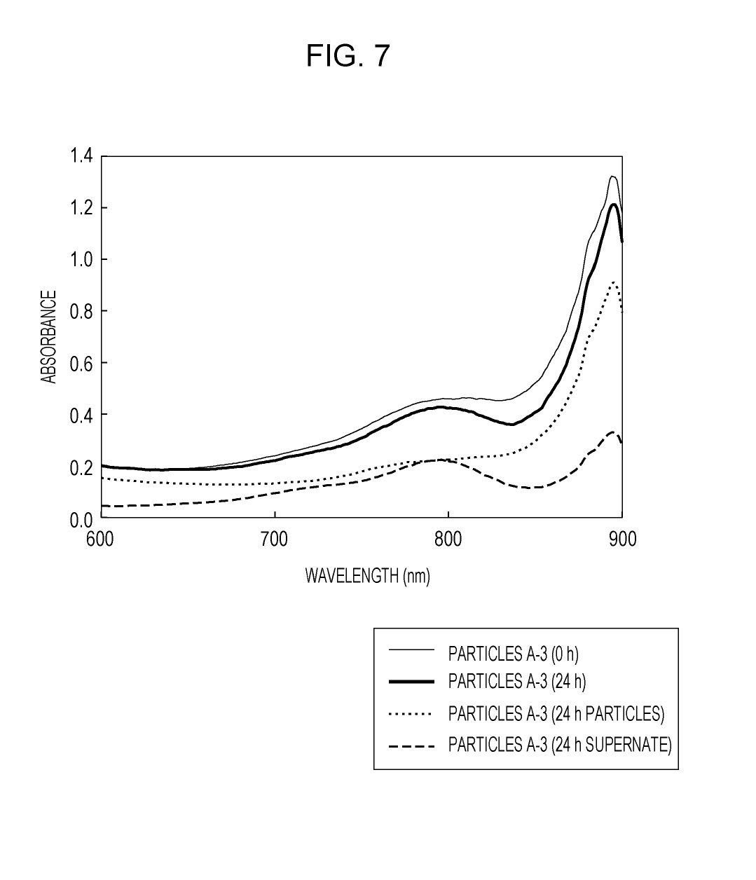

After the absorbance was determined again, the mixture was separated into particles and a supernate using a magnet in particles A-1 to A-3 and using a magnetic column in particles A-4. The absorbance of the aqueous dispersion of the particles and the absorbance of the supernate were determined in order to check the presence of ICG adsorbed on the iron oxide particles and the presence of the ICG that was desorbed from the iron oxide particles and leaked into the supernate. FIG. 7 shows the results of the measurement of the absorbance of particles A-3. In FIG. 7, “0 h” refers to the absorbance determined immediately after mixing the particles with blood serum; “24 h” refers to the absorbance of an aqueous dispersion of the particles after the particles were incubated for 24 hours in blood serum; “24 h particles” refers to the absorbance of the particles having been incubated for 24 hours in blood serum; and “24 h supernate” refers to the absorbance of the supernate obtained after incubating the particles for 24 hours in blood serum.

FIG. 7 shows that the relative maximum optical absorption observed at 895 nm was maintained even after incubating a mixture of particles A-3 and blood serum for 24 hours.

The ratio of ICG that was not desorbed from but adsorbed on the particles after the particles were incubated for 24 hours in blood serum may be represented by the following index:
(ratio of ICG remaining in blood serum)=(absorbance of particles after incubation for 24 hours)/(absorbance of particles immediately after mixing with blood serum)×100(%). The higher the index is, the greater the number of ICG molecules that are not desorbed from the iron oxide particles is, that is, the greater the number of ICG molecules that are adsorbed on the iron oxide particles.

In particles A-3, 75% of ICG was presumably adsorbed on the iron oxide particles.

For particles A-1, A-2, and A-4, the ratio of ICG remaining in blood serum was determined as in the evaluation of particles A-3. Table summarizes the data of particles according to Examples.

TABLE

| | Particles | Particles | Particles | Particles | Particles | Particles | Particles |
|---|---|---|---|---|---|---|---|
| | B-1 | B-2 | B-3 | A-1 | A-2 | A-3 | A-4 |
| Wavelength at | 873 nm | 852 nm | 809 nm | 895 nm | 895 nm | 895 nm | 895 nm |
| which relative | | | | | | | |
| maximum | | | | | | | |
| absorbance is | | | | | | | |
| observed | | | | | | | |
| Ratio of | 1.1 | 0.8 | 0.3 | 3.2 | 3.1 | 2.6 | 2.7 |
| absorbance at | | | | | | | |
| 895 nm to that at | | | | | | | |
| 780 nm | | | | | | | |
| Ratio of ICG | 0% | 0% | 0% | 40% | 73% | 75% | 56% |
| remaining in | | | | | | | |
| blood serum | | | | | | | |

Table shows that, in the particles according to Examples, the ratio of ICG remaining in blood serum was high and ICG was less likely to be desorbed from the iron oxide particles.

### Measurement of Intensity of Photoacoustic Signal of Particles A-3

The intensity of the photoacoustic signal of particles A-3 was determined as follows.

A photoacoustic signal was measured by irradiating particles dispersed in water with a laser pulse, detecting a photoacoustic signal generated from the particles using a piezoelectric element, amplifying the photoacoustic signal with a high-speed preamplifier, and capturing a waveform with a digital oscilloscope. The detailed measurement conditions are as follows. A laser pulse source was a titanium-sapphire laser (LT-2211-PC, produced by LOTIS Ltd.) with a wavelength of 900 nm, an energy density of 20 to 50 mJ/cm² (the energy density varies depending on the selected wavelength), a pulse duration of about 20 nanoseconds, and a pulse-repetition rate of 10 Hz. A measurement container used for storing the particles dispersed in water was a polystyrene cuvette having a width of 1 cm and an optical path length of 0.1 cm. The piezoelectric element used for detecting the photoacoustic signal was a non-focusing ultrasonic transducer (Panametrics-NDT, V303) having an element diameter of 1.27 cm and a center frequency of 1 MHz. The measurement container and the piezoelectric element were immersed in a glass container filled with water with a spacing of 2.5 cm between the measurement container and the piezoelectric element. The high-speed preamplifier used for amplifying the photoacoustic signal was an ultrasonic preamplifier (Model 5682, produced by Olympus Corporation) with an amplification degree of +30 dB. The amplified signal was sent to a digital oscilloscope (DPO4104, produced by Tektronix, Inc.). The polystyrene cuvette was then irradiated with a laser pulse from the outside of the glass container. A part of the scattered light generated due to the irradiation was detected with a photodiode and then sent to the digital oscilloscope as a trigger signal. The digital oscilloscope was set to Average Mode for displaying the average signal of 32 observations, and the average of photoacoustic signals generated due to 32 laser pulse irradiations was determined. The intensity (V) of the photoacoustic signal was determined on the basis of the waveform of the average photoacoustic signal. The value obtained by dividing the intensity of the average photoacoustic signal by the power (J) of the pulse laser with which the particles were irradiated was defined as a normalized photoacoustic signal (VJ⁻¹).

Particles A-3 were dissolved by hydrochloric acid, and subsequently the Fe concentration was determined by colorimetry using bathophenanthroline-disulfonic acid.

The normalized photoacoustic signal was divided by the Fe concentration to obtain a normalized photoacoustic signal per mole of iron PA (Fe) (VJ⁻¹M⁻¹). The normalized photoacoustic signal per amount of iron PA (Fe) of particles A-3 was 2.8×10⁻⁷ (VJ⁻¹M⁻¹).

### Comparative Example 1

### Preparation of Particles Each Including Iron Oxide Particle and ICG (Particles B-1 and B-2)

Particles each including an iron oxide particle and ICG (particles B-1 and B-2) were prepared as Comparative examples as follows. The iron oxide particles and ICG used as raw materials were the same as those used for preparing particles A-1, A-2, and A-3.

Water was added to ICG to prepare a liquid mixture having an ICG concentration of 1.29 mg/ml. The liquid mixture was then irradiated with an ultrasonic wave for 10 minutes to dissolve ICG in water. The resulting aqueous solution of ICG was mixed with an aqueous solution in which iron oxide particles are dispersed in a volumetric ratio of 9:1 to prepare a mixture. The resulting mixture was divided into 2 portions, which were then heated at 65° C. and 37° C. for 24 hours. The iron oxide particles were collected using a magnet to remove ICG that was not adsorbed on the iron oxide particles. The particles heated at 65° C. and 37° C. were denoted as B-1 and B-2, respectively.

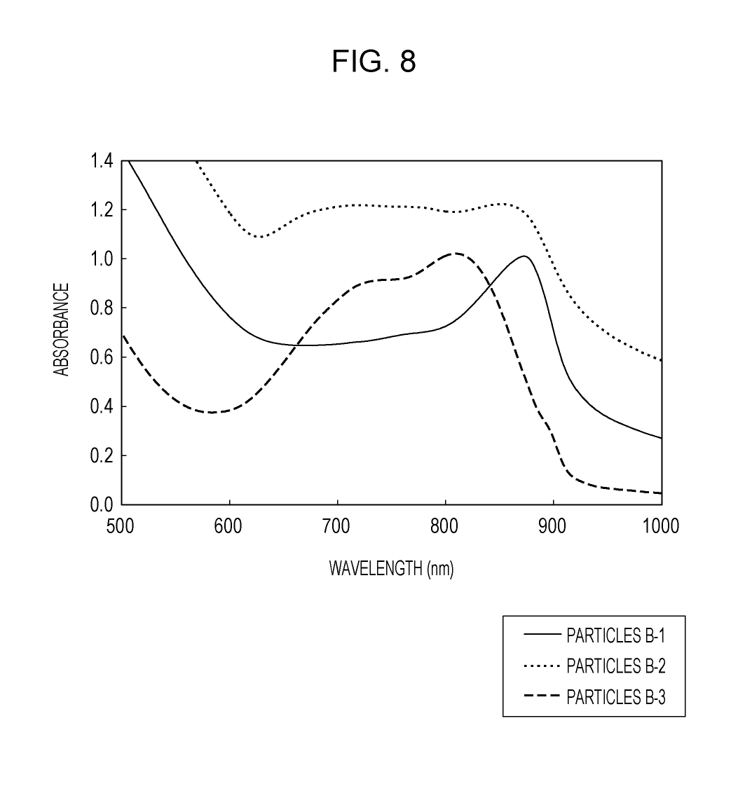

The absorbances of particles B-1 and B-2 were determined as in Example 1 (FIG. 8). The relative maximum absorbance was observed at 873 nm in particles B-1 and at 852 nm in particles B-2. The ratio of the absorbance at 895 nm to the absorbance at 780 nm was 1.1 in particles B-1 and 0.8 in particles B-2.

The results show that it is impossible to produce particles having a relative maximum absorbance at 880 nm or more and 910 nm or less without heating an aqueous solution of ICG prior to mixing the iron oxide particles with the aqueous solution of ICG.

### Comparative Example 2

Preparation of Particles Each Including Iron Oxide Particle and ICG with Dispersant on Surface of Particle (Particles B-3)

Particles each including an iron oxide particle and ICG with dextran serving as a dispersant on surface of the particle (particles B-3) were prepared. The particles were prepared as in the preparation of particles B-1, except that iron oxide particles (Nanomag 79-00-501, particle size 50 nm, γ-Fe₂O₃, produced by Corefront Corporation) having dextran on the surfaces of the particle were used instead of the iron oxide particles used for preparing particles B-1 (Nanomag 45-00-202, γ-Fe₂O₃, produced by Corefront Corporation). The resulting particles were denoted as B-3.

The absorbance of particles B-3 was determined as in Example 1 (FIG. 8). Particles B-3 had a relative maximum absorbance at 809 nm, and the ratio of the absorbance at 895 nm to the absorbance at 780 nm was 0.3.

The results show that it is impossible to produce particles having a relative maximum absorbance at 880 nm or more and 910 nm or less without heating an aqueous solution of ICG prior to mixing the iron oxide particles with the aqueous solution of ICG.

### Evaluation of Particles B-2 in Terms of Stability in Water

Particles B-2 was evaluated in terms of stability in water as in Evaluation of Particles A-3 in Terms of Stability in Water. The absorbance due to ICG contained in particles B-2 was determined at a wavelength of 852 nm.

FIG. 6B shows the absorbance change ratio of particles B-2. It was found that, in particles B-2, which had a large absorbance change ratio, ICG was likely to be desorbed from the iron oxide particles in water.

### Evaluation of Particles B-1 to B-3 in Terms of Stability in Blood Serum

As a model experiment for demonstrating the administration of particles B-1 to B-3 into a living body, particles B-1 to B-3 were each mixed with blood serum and the ratio of ICG remaining in blood serum was determined as in Evaluation of Particles A-1 to A-4 in Terms of Stability in Blood Serum. Particles and a supernate were separated using a magnet in particles B-1 and B-2 and using a magnetic column in particles B-3. Table shows the results. The results show that, in particles that do not have a relative maximum absorbance at 880 nm or more and 910 nm or less, ICG may be desorbed from iron oxide particles in blood serum.

In the indocyanine green-containing particles according to the invention, which each include a metal oxide particle or a metal particle and an aggregate of ICG, ICG is less likely to be desorbed from the metal oxide particle or the metal particle even when the particles are placed in water or blood serum.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-138393 filed Jun. 20, 2012, which is hereby incorporated by reference herein in its entirety.

What is claimed is:

1. Indocyanine green-containing particles each comprising:
a metal oxide particle or a metal particle; and
an aggregate of indocyanine green,
the aggregate of indocyanine green having a relative maximum absorbance at 880 nm or more and 910 nm or less.

2. The indocyanine green-containing particles according to claim 1,
wherein the aggregate of indocyanine green is adsorbed on the metal oxide particle or the metal particle.

3. The indocyanine green-containing particles according to claim 1,
wherein the metal oxide particle is an iron oxide particle.

4. The indocyanine green-containing particles according to claim 1,
wherein the absorbance of the indocyanine green-containing particles at 895 nm is 2.0 times or more the absorbance at 780 nm.

5. The indocyanine green-containing particles according to claim 1,
wherein the indocyanine green-containing particles each include a dispersant on the surface thereof.

6. The indocyanine green-containing particles according to claim 5,
wherein the dispersant is dextran.

7. A photoacoustic-imaging contrast agent comprising:
the indocyanine green-containing particles according to claim 1; and
a dispersion medium.

8. A method for producing the indocyanine green-containing particles according to claim 1, the method comprising the steps of:
heating an aqueous solution of indocyanine green;
mixing the heated aqueous solution of indocyanine green with metal oxide particles or metal particles to prepare a liquid mixture; and
heating the liquid mixture.
